(19) United States
(12) Patent Application Publication
(10) Pub. No.: US 2008/0027380 A1
(43) Pub. Date: Jan. 31, 2008

(54) VASCULAR ACCESS DEVICE AND METHOD

(76) Inventors: Mark H. Wholey, Oakmont, PA (US); Michael H. Wholey, San Antonio, TX (US)

Correspondence Address: PIETRAGALLO, BOSICK & GORDON LLP, ONE OXFORD CENTRE, 38TH FLOOR, 301 GRANT STREET, PITTSBURGH, PA, 15219-6404, US

(21) Appl. No.: 11/830,915

(22) Filed: Jul. 31, 2007

Related U.S. Application Data
(60) Provisional application No. 60/834,400, filed on Jul. 31, 2006.

(51) Int. Cl. A61M 29/00

(52) U.S. Cl. 604/104

(57) ABSTRACT

A vascular access device capable of provided entry into a blood vessel with minimal trauma and a minimal number of steps is disclosed. In one embodiment, the device utilizes 21-guage needle to install a guide wire that is 0.018 inch in diameter. A 5-8 French outer sheath and inner cannula are closely dimensioned to the guide wire to improve tracking along the guide wire. The inner cannula may also have an annular recess at a tapered distal end. An annular protrusion is then provided on the distal tip of the outer sheath. The annular protrusion seats in the annular recess to provide a smooth exterior surface.

### CROSS REFERENCE TO RELATED APPLICATION

This application claims the benefit of U.S. Provisional Patent Application Ser. No. 60/834,400 filed Jul. 31, 2006, which is herein incorporated by reference.

### FIELD OF THE INVENTION

This invention relates medical devices and methods, and particularly, to devices and methods for providing vascular access for medical sheaths and guiding catheters.

### BACKGROUND INFORMATION

Arterial and venous vascular access is necessary for a wide array of medical purposes. Intra-vascular techniques include angioplasty, artherectomy and endovascular aneurysm repair.

A wide variety of vascular access techniques exist. However, many use a common series of steps. Initial access to a blood vessel may be provided by a puncture with a 19-gauge needle. A 0.035-inch (0.0889 centimeter) guide wire is then inserted through the needle and the needle is removed. A 4 to 8 French vascular sheath with inner dilator is then inserted over the 0.035-inch (0.0889 centimeter) guide wire. The inner dilator is removed and other devices may be installed. The relatively large size of the needle in this procedure, however, made this conventional technique too traumatic for some applications. In other applications, involving smaller blood vessels, it is nearly impossible to gain access with a 19-gauge needle.

Micropuncture techniques developed to allow for easier and less traumatic vascular access. The micropuncture techniques utilize a 21-gauge needle to make the initial puncture of the tissue and blood vessel. A 0.018-inch (0.0457 centimeter) guide wire is then inserted through the 21-gauge needle and the needle is removed. A coaxial outer sheath including an inner and outer dilator is then advanced over the 0.018-inch (0.0457 centimeter) guide wire and the inner dilator along with the guide wire is then removed. Next, a 0.035-inch (0.0889 centimeter) wire may then be inserted through the coaxial outer sheath and the outer dilator may then be removed. A 5 to 8 French outer sheath is then inserted over the 0.035-inch (0.0889 centimeter) guide wire and the guide wire is removed. Other devices may then access the blood vessel through the vascular outer sheath. While the micropuncture technique lessens the traumatic effect of the initial vascular access and allows access in difficult access cases, it suffers from requiring an excessive amount of procedural steps. The extra steps increase trauma to the puncture area and also increase to time required to complete the procedure.

The present invention has been developed in view of the foregoing.

### SUMMARY OF THE INVENTION

A vascular access device capable of provided entry into a blood vessel with minimal trauma and a minimal number of steps is disclosed. In one embodiment, the device utilizes 21-guage needle to install a guide wire that is 0.018 inch (0.0457 centimeter) in diameter. A 5-8 French outer sheath and inner cannula are closely dimensioned to the guide wire to improve tracking along the guide wire. The inner cannula may also have an annular recess at a tapered distal end. An annular protrusion is then provided on the distal tip of the outer sheath. The annular protrusion seats in the annular recess to provide a smooth exterior surface.

An aspect of the present invention provides a vascular access device comprising a small caliber guide wire, an inner cannula coaxially located about the small caliber guide wire comprising an exterior having an inwardly tapered distal end and an inwardly extending annular recess adjacent the tapered distal end and an outer sheath coaxially located about the inner cannula comprising a lumen having an inwardly extending annular protrusion at a distal end of the outer sheath which seats within the annular recess of the inner cannula.

Another aspect of the present invention provides a vascular access device comprising a small caliber guide wire, an inner cannula coaxially located about the small caliber guide wire comprising an exterior having an inwardly extending tapered distal end and an inwardly extending first annular recess adjacent the tapered distal end, an outer cannula coaxially located about the inner cannula having a tapered distal end having an inwardly extending first annular protrusion at a distal tip of the tapered distal end and an inwardly extending second annular recess adjacent the tapered distal end an outer sheath coaxially located about the inner cannula comprising a lumen having a second annular protrusion at a distal end of the outer sheath wherein the first annular protrusion of the outer cannula seats with the first annular recess of the inner cannula and the second annular protrusion of the outer sheath seats with second annular recess of the outer cannula.

Another aspect of the present invention provides a vascular access device comprising an outer sheath, a small caliber wire inserted into a blood vessel, means for inserting the outer sheath having a diameter of at least 5 French along the small caliber guide wire into a blood vessel.

A further aspect of the present invention provides a kit containing a vascular access device consisting of a guide wire having a diameter less or equal to 0.018 inch (0.0457 centimeter), an inner cannula, an outer sheath and a 21 gauge needle.

Yet another aspect of the present invention provides A kit containing a vascular access device consisting of a guide wire having a diameter less or equal to 0.018 inch (0.0457 centimeter), an inner cannula, an outer cannula, an outer sheath and a 21 gauge needle.

Another aspect of the present invention provides a method of installing a vascular access device in blood vessel of a patient comprising the steps of inserting a 21 gauge needle into a blood vessel of a patient, inserting a guide wire through the needle and partly into the blood vessel, removing the needle, inserting an inner cannula coaxially fitted with an outer sheath into the blood vessel until a portion of the outer sheath is within the blood vessel and removing the inner cannula and guide wire.

These and other aspect will become more apparent from the following description.

### BRIEF DESCRIPTION OF THE DRAWINGS

FIG. 1 is a vascular access device inserted through the skin and into the femoral artery of a patient according to one embodiment of the present invention.

FIG. 2 is a side view of a vascular access device inserted within a blood vessel showing a sectional view of the blood vessel and surrounding tissue according to one embodiment of the present invention.

FIG. 3 is a longitudinal oblique view of a vascular access device having a single inner cannula according to one embodiment of the present invention.

FIG. 4 is an expanded longitudinal cross sectional view of the distal end of the vascular access device of FIG. 3 according to one embodiment of the present invention.

FIG. 5 contains four longitudinal oblique views of the various components of the device shown in FIG. 3.

FIG. 6 shows a cross-section of the components shown in FIG. 5.

FIG. 7 shows an oblique view of the various components of the vascular access device having two inner cannulas shown in FIG. 2 according to one embodiment of the present invention.

FIG. 8 shows longitudinal cross-sectional view of the components shown in FIG. 7 according to one embodiment of the present invention.

FIG. 9 is an expanded longitudinal cross sectional view of the distal end of the vascular access device of FIG. 1 according to one embodiment of the present invention.

FIG. 10 is a longitudinal oblique view of a vascular access device having a single inner cannula made of metal with a partial cut away portion of the inner cannula according to one embodiment of the present invention.

### DETAILED DESCRIPTION

For purposes of the following detailed description, it is to be understood that the invention may assume various alternative variations and step sequences, except where expressly specified to the contrary. Moreover, other than in any operating examples, or where otherwise indicated, all numbers expressing, for example, dimensions used in the specification and claims are to be understood as being modified in all instances by the term “about”. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements and manufacturing processes.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

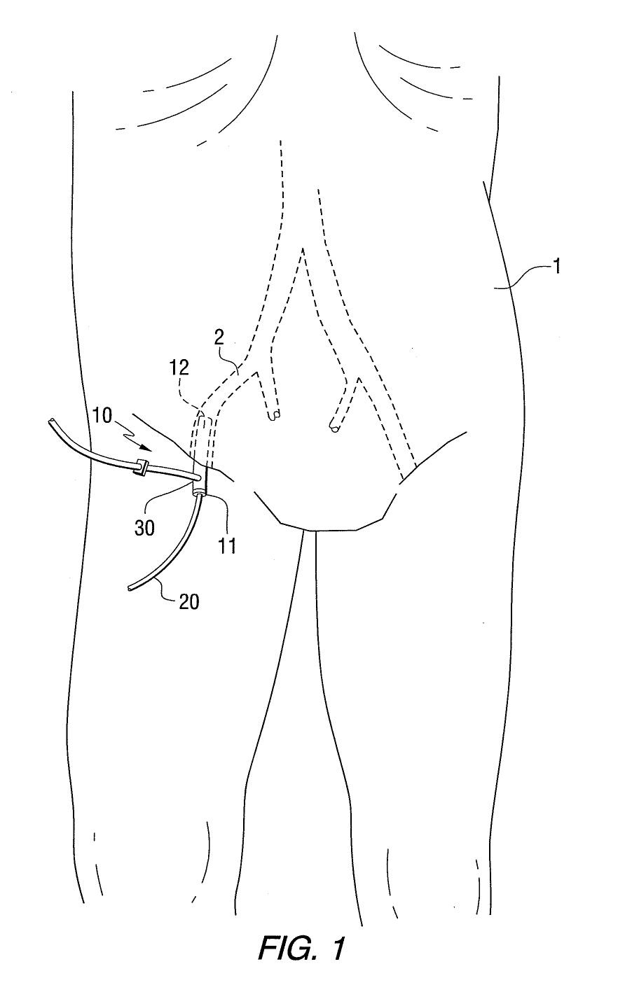

Referring now to FIG. 1, a patient 1 is shown with a vascular access device 10 installed within a blood vessel 2, represented in this figure as a patient's femoral artery. When installed, a device may have a small caliber guide wire 20 coaxially coursing through an outer sheath 30. The vascular access device 10 has a proximal end 11 and a distal end 12.

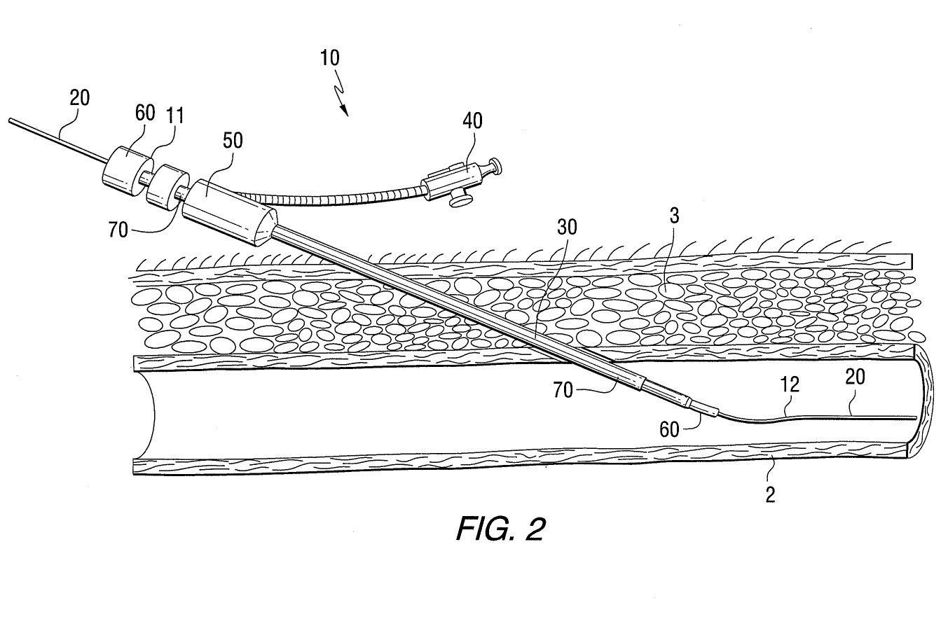

Referring now to FIG. 2, a side view of a vascular access device 10 is shown through a sectional view of tissue layer 3 and blood vessel 2. The proximal end 11 of the vascular access device 10 includes one or more connections well known in the art. For example, luer end 50 connects the outer sheath 30 with the outer cannula 70 and flush bag line 40. The flush bag line 40 is used for introducing and aspirating fluids through the vascular access device 10. In this embodiment, the vascular access device 10 has an inner cannula 60 and an outer cannula 70. The outer cannula 70 is coaxially and slidably located within the outer sheath 30. The inner cannula 60 is coaxially and slidably located within the outer cannula 70. Coaxially and slidably coursing through the inner cannula 60 is a small caliber guide wire 20.

The outer sheath 30 structured and arranged to allow vascular access for medical devices such as diagnostic catheters, guidewires, guiding catheters, interventional equipment such as percutaneous transluminal angioplasty balloon catheters, intravascular stents, cutting balloon catheters and other devices depending on the diseased vessel segment. This outer sheath 30 can also provide access into venous structure as well as non-vascular structures in the renal and biliary system. The outer is 4-8 French in size and may be for example, 5-85 cm (33.5 inches) in length. As mentioned above, one advantage of the present invention is the ability to transition from a small caliber guide wire to a 4-8 French outer sheath. As used herein small caliber refers to less than or equal to the nominal inside diameter of a 21 gauge needle. The small caliber guide wire 20 may be 0.014 inch (0.0356 centimeter) to 0.0195 inch (0.0495 centimeter) ±0.001 inch (−0.00254 centimeter) in diameter, for example, 0.018 inch (0.0457 centimeter) ±0.001 inc, and may have a length of for example, 25 cm (9.84 inches) −300 cm (118 inches). This thin wire can then be used in the small 21 gauge needle to gain access into the blood vessel 2. A 21 gauge needle provides many advantages over a larger bore needle, e.g. better patient comfort, fewer complications from dissections, hemoatoma, and other adverse events, and better access to smaller, more difficult to reach blood vessels. The small caliber guide wire 20 may be made of stainless steel or of a nickel-titanium alloy, for example, an alloy identified by the trade name Nitinol. The small caliber guide wire 20 may also have a platinum tip for providing safe and visible access to the blood vessel 2. The guidewire is designed to have a 1-1 torque capability to enable passage through difficult atherosclerotic vessels. The small caliber guide wire 20 also has a smooth transition from the Nitinol core to the platinum tip providing the ability to maintain a slightly curved tip without dissecting tissue planes.

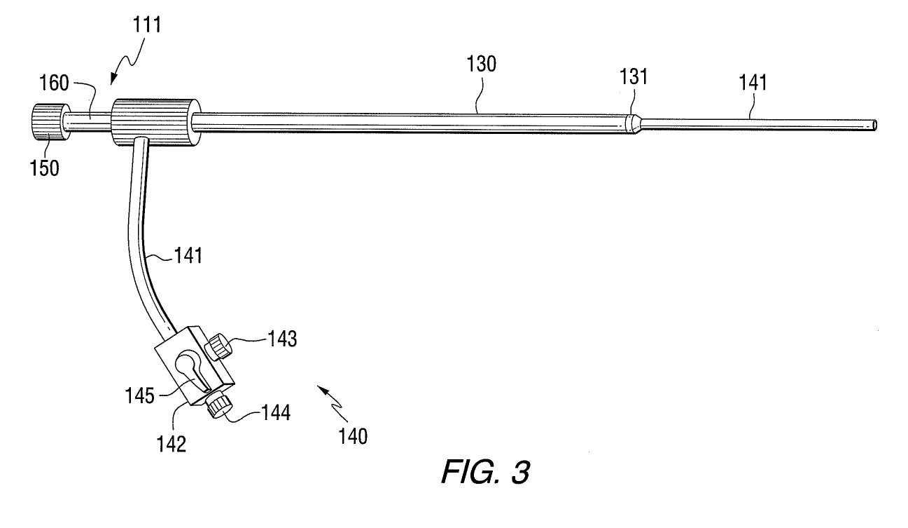

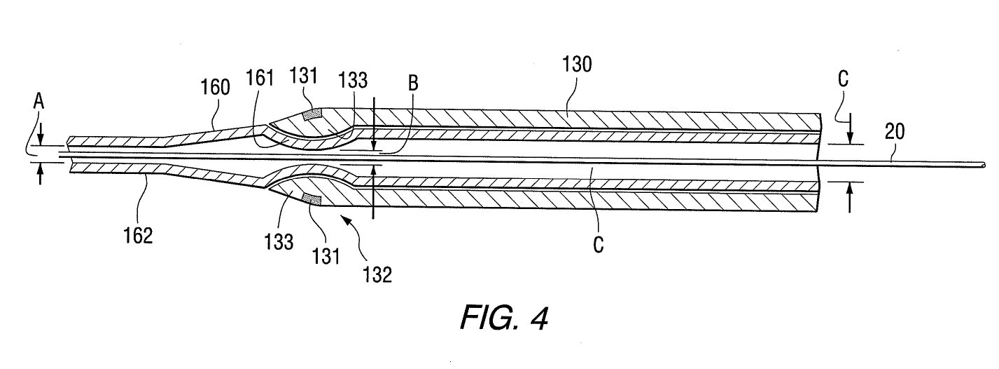

Referring now to FIG. 3-4, a vascular access device 100 is shown according to one embodiment of the present invention. In this embodiment, the device 100 has a single inner cannula 160. The inner cannula 160 inserts into a luer end 150 at a proximal end 111 of the outer sheath 130. The inner cannula 160 courses through the outer sheath 130. The inner cannula 160 has a tapered distal end 161 which exits a distal portion of the outer sheath 130 which may be equipped with a radiopaque marker 131. Extending from luer end 150 is a tube 141 of flush bag line 140. Tube 141 is connected at one end to the proximal 111 of the vascular access device 100 and at the other end to a valve assembly 142 such as a stopcock. The valve assembly 142 may have side ports 143, 144 for attaching introduction and aspiration of fluids and may also have a switch 145 for selecting said side ports 143, 144.

Referring now to FIG. 4, an expanded cross section of the distal end of the vascular access device 100 of FIG. 3 is shown according to one embodiment of the present invention. As shown in FIG. 4, the inner cannula 160 has an annular recess 161 adjacent the tapered distal end of the inner cannula 160. Also shown in FIG. 4 is the outer sheath 130 having a radiopaque marker 131 inserted in a distal end 132 of the outer sheath 130. Radially inwardly projecting from the distal end 132 of the outer sheath 130 is an annular protrusion 133. The annular protrusion 133 is congruently shaped to fit within the annular recess 161 of the inner cannula 160. The fit of the annular recess 161 and annular protrusion 133 provides for a smooth tapered profile about the exterior of the vascular access device 100 as it transitions from inner cannula 160 to the outer sheath 130. The smooth profile reduces trauma to the surrounding tissue as the combination of inner cannula 160 and outer sheath 130 installed along the small caliber guide wire 20.

The annular recess 161 of the inner cannula 160 also aides in providing a tight tolerance for the inner cannula 160 to the small caliber guide wire 20. The inner cannula 160 has an inside diameter at A of about 0.019 inch (about 0.0483 centimeter) to about 0.021 inch (about 0.0533 centimeter) for example, 0.018 inch (0.0457 centimeter). Similarly, the inner cannula has an inside diameter at B of about 0.019 inch (about 0.0483 centimeter) to about 0.021 inch (about 0.0533 centimeter) for example, 0.018 inch (0.0457 centimeter). The remainder of the inner cannula has an inside diameter of about 0.021 inch (about 0.0533 centimeter) to about 0.026 inch (about 0.066 centimeter) for example, 0.023 inch (0.0584 centimeter). The extremely close tolerance of the inner diameter of the inner cannula 160 to small caliber guide wire 20. This close tolerance enables the vascular access device 100 to be inserted along the small caliber guide wire 20 without kinking. The annular recess 161 and tapered end 162 achieve even closer tolerances in their respective areas further enabling the transition from small diameter small caliber guide wire 20 to the inner cannula 160 to the outer sheath 130.

In a preferred embodiment, the guide wire is coated with a friction reducing substance, such as polytetraflouroethylene (PTFE often sold under the trademark Teflon®). In this embodiment, the I.D. of the the inner cannula at B is within 0.002 of the target O.D. of the guide wire.

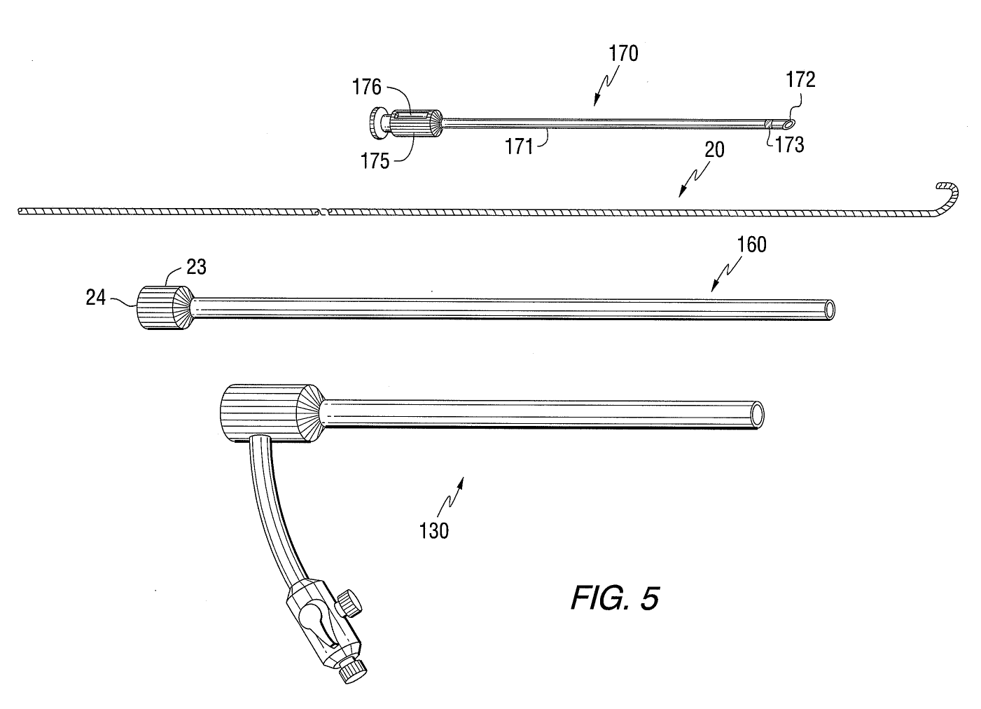

A kit containing the various components of a vascular access device 100 is shown in FIG. 5, according to one embodiment of the present invention. The outer sheath 130, inner cannula 160 and small caliber guide wire 20 are shown disassembled. An additional component is included with the kit in FIG. 5. The additional component is a percutaneous needle 170 used to make the initial tissue and blood vessel puncture. The needle 170 is a 21-gauge needle. The needle 170 may come in varying lengths for example, 5, 7 and 9 cm (2.76 and 3.54 inches) lengths. The needle 170 may also include a beveled edge 172 with ecogenic capabilities to see the tip under ultrasound guidance, if needed. The hub 175 of the needle 170 may have a small notch 174 on or near the hub 175 of the needle. The notch 174 indicates the rotational position of the beveled edge 172 along a longitudinal axis of the needle 170 once the needle 170 has been inserted into the patient 1.

A vascular access device of the present invention may be utilized in the following manner. After anesthesia has been provided to the skin site, the blood vessel is accessed with a 21-gauge needle 170. A 0.018 inch (0.0457 centimeter) guide wire is then inserted through the needle 170 and into the blood vessel 2. The needle 170 is then removed. The combination of inner cannula 160 and sheath 130 is then slid over the small caliber guide wire 20 until a distal portion of the 4-8 French outer sheath is positioned within the blood vessel 2. The small caliber guide wire 20 and inner cannula 160 may then be removed allowing access for other medical devices.

It should be noted that the cannulas 60, 70, 160 and outer sheaths 30, 130 of the present invention may be made of a durable plastic for example; braided and non-braided polyethylene, polyurethane, and Teflon® could all be used. These materials provide the stiffness and rigidity necessary to help the cannulas and outer sheaths gain access through the tough and difficult tissue planes found in patient with deep or scarred groins. The rigidity of this material coupled with a close tolerance between the cannula is one reason a narrow cannula, and thin guide wire can be used to install a 5 to 8 French outer sheath. In one embodiment, the cannulas and/or outer sheath is cross wound with small caliber wire within the wall to further increase rigidity.

Referring again to FIG. 4, the congruently shaped annular recess 161 and annular protrusion 133 have a generally radial or generally elliptical shape. This shape and fit provides resistance to co-axial movement when the annular protrusion 133 is seated within the annular recess 161 and provides a smooth, tapered transition between the exterior surface of the inner cannula 160 and the exterior surface of the outer sheath 130. However, co-axial movement is not prevented. As noted, the inner cannula 160 must be able to be removed from the outer sheath 130 once the outer sheath 130 is positioned within the blood vessel 2. The generally radial or generally elliptical configuration of the annular protrusion 133 and annular recess 161 permits co-axial movement of the outer sheath 130 and inner cannula 160.

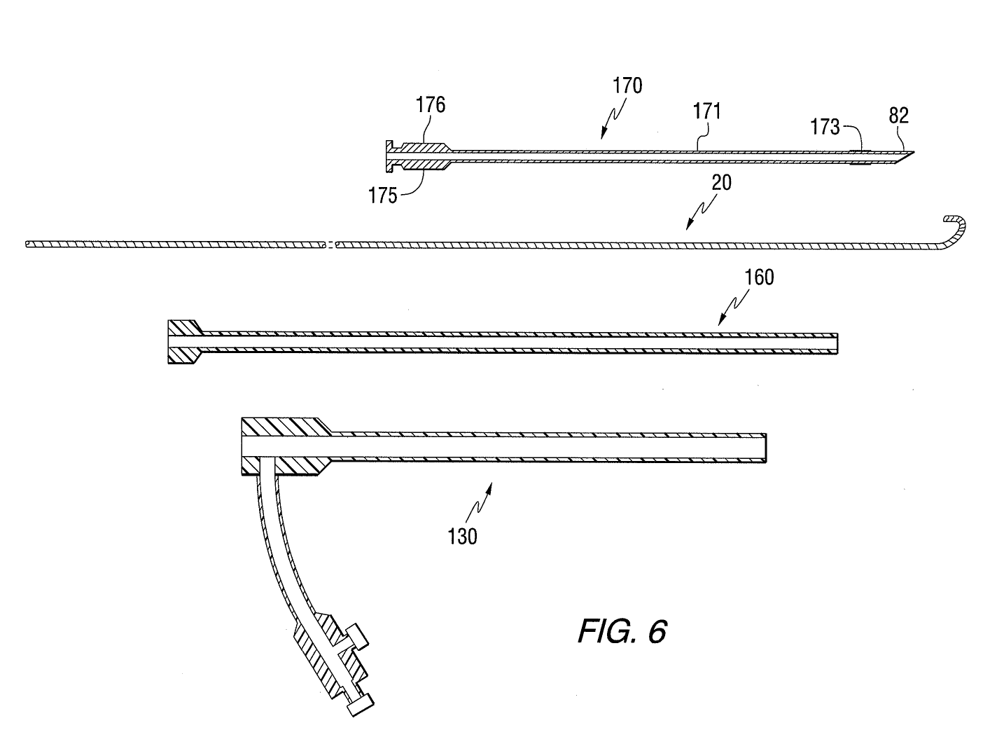

FIG. 6 is a cross section of the components shown in FIG. 5. The needle 170 is shown with a main shaft 171 and beveled distal tip 172 equipped with a radiopaque marker 173. The proximal end 174 of the needle 170 may have a luer end or hub 175 through which the small caliber guide wire 20 may pass to be installed in the blood vessel 2.

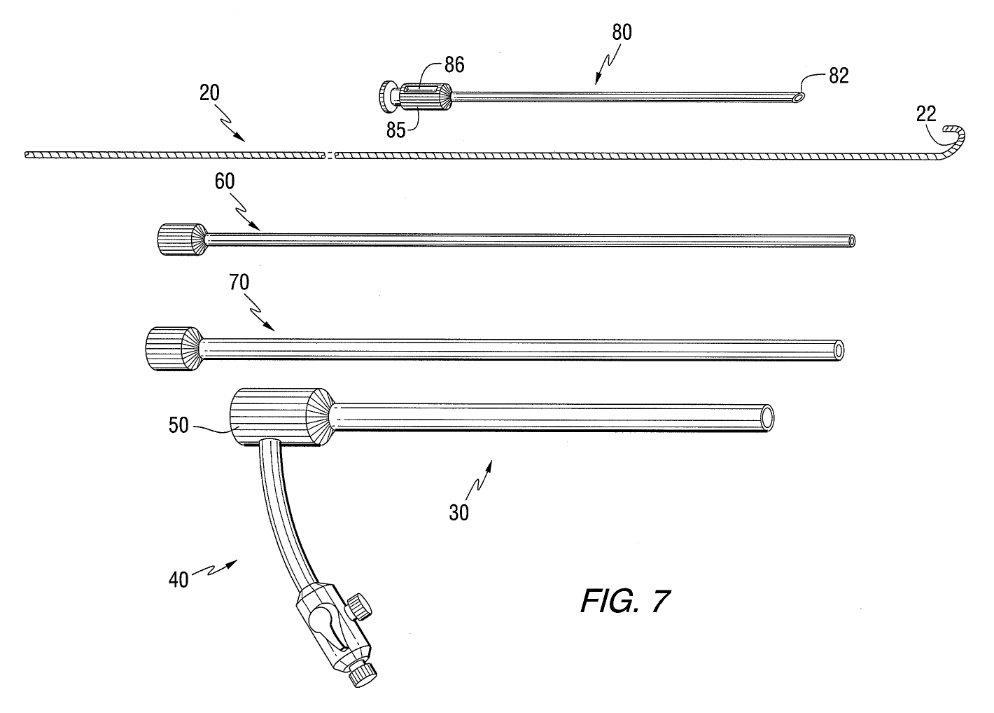

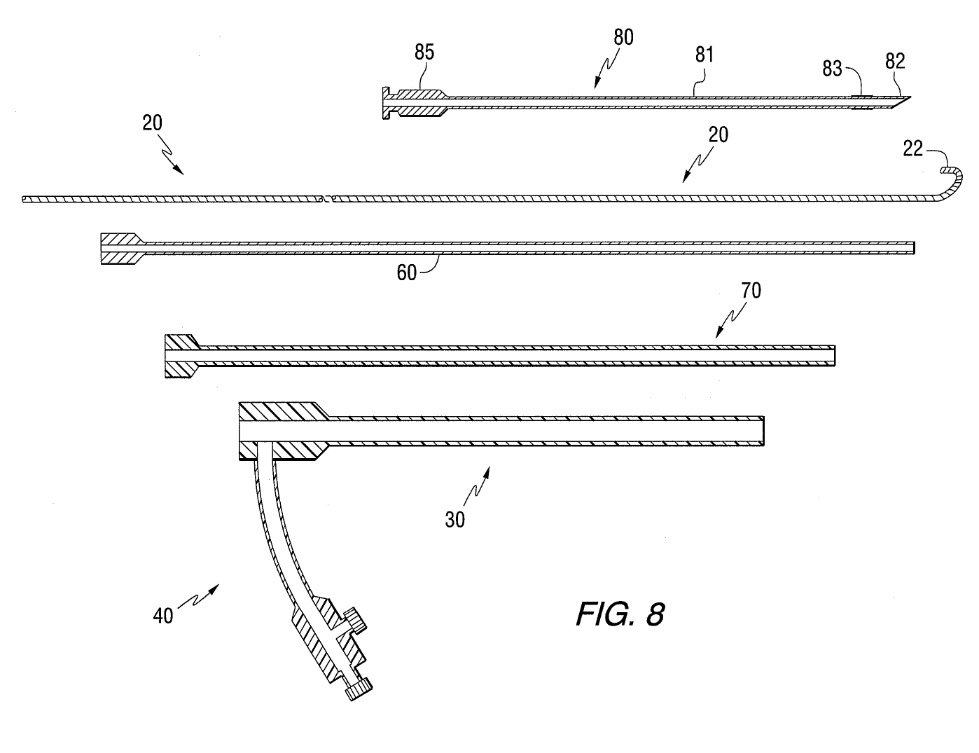

FIG. 7 shows the disassembled components which may be found in a kit for the vascular access device 10 of FIG. 2 according to one embodiment of the present invention. As seen the kit may contain a needle 80, a small caliber guide wire 20 a curved distal tip 22, an inner cannula 60, an outer cannula 70, an outer sheath 30 and a flush bag line 40 connected to the outer sheath 30 through luer end 50. Like the needle 170 described above, the needle 80 may have a main shaft 81, a beveled end 82 with a radiopaque marker 83 and a hub 86 with a notch 86. FIG. 8 shows a cross section of the individual components shown in FIG. 7.

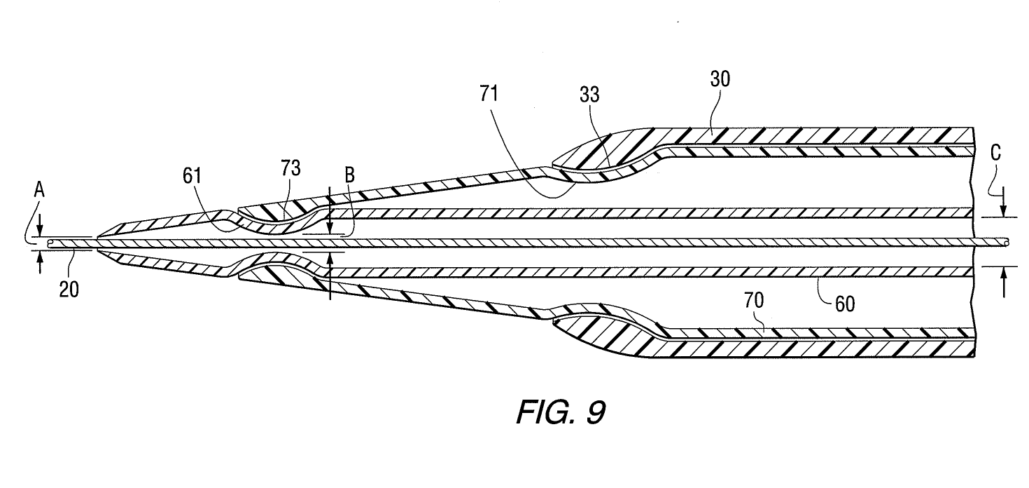

As seen in FIG. 9, an assembled cross sectional view of an embodiment of the present invention having an inner cannula 60, an outer cannula 70 and an outer sheath 30 is shown. Similarly to a single cannula embodiment the outer sheath 30 may have an annular protrusion extending radially inward at its distal end. However, in this embodiment the annular protrusion 33 will seat in an annular recess 71 of the outer cannula 70. The outer cannula 70 may then have an annular protrusion 73 which seats in the annular recess 61 of the inner cannula 60. The inside diameters at positions A, B and C are dimensioned as described above in reference to FIG. 4.

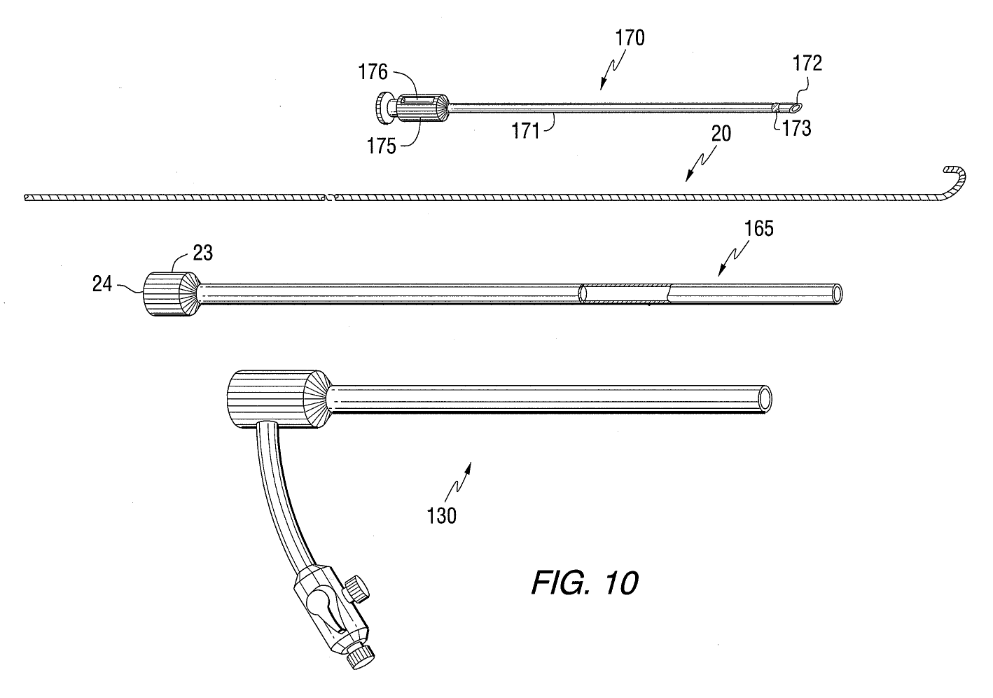

For extremely scarred groins, inner cannulas 165 made of metal may be provided to achieve greater support than that achieved by traditional vascular access devices as seen in FIGS. 10-11. Metals used for the inner cannula 165 may include stainless steel and titanium including alloys of each.

Whereas particular embodiments of this invention have been described above for purposes of illustration, it will be evident to those skilled in the art that numerous variations of the details of the present invention may be made without departing from the invention as defined in the appended claims.

1. A vascular access device comprising:
a small caliber guide wire;
an inner cannula coaxially located about the small caliber guide wire comprising an exterior having an inwardly tapered distal end and an inwardly extending annular recess adjacent the tapered distal end; and
an outer sheath coaxially located about the inner cannula comprising a lumen having an inwardly extending annular protrusion at a distal end of the outer sheath which seats within the annular recess of the inner cannula.

2. A vascular access device of claim 1, wherein the guide wire has a diameter of less than or equal to 0.018 inch.

3. A vascular access device of claim 1, wherein the inner cannula has a first inside diameter at a distal tip of the annular recess and a second inside diameter along at least a portion of the remainder of the inner cannula.

4. A vascular access device of claim 3, wherein the first inside diameter of the inner cannula is between 0.019 inch and 0.021 inch.

5. A vascular access device of claim 3, wherein the second inside diameter of the inner cannula is between 0.021 inch and 0.026 inch.

6. A vascular access device according to claim 1, wherein the guide wire is coated with a PTFE.

7. A vascular access device according to claim 1, wherein the annular recess and annular protrusion are generally radius ed or generally elliptical along a longitudinal direction.

8. A vascular access device comprising:
a small caliber guide wire;
an inner cannula coaxially located about the small caliber guide wire comprising an exterior having an inwardly extending tapered distal end and an inwardly extending first annular recess adjacent the tapered distal end;
an outer cannula coaxially located about the inner cannula having a tapered distal end having an inwardly extending first annular protrusion at a distal tip of the tapered distal end and an inwardly extending second annular recess adjacent the tapered distal end; and
an outer sheath coaxially located about the inner cannula comprising a lumen having a second annular protrusion at a distal end of the outer sheath;
wherein the first annular protrusion of the outer cannula seats with the first annular recess of the inner cannula and the second annular protrusion of the outer sheath seats with second annular recess of the outer cannula.

9. A vascular access device of claim 8, wherein the guide wire has a diameter of less than or equal to 0.018 inch.

10. A vascular access device according to claim 8, wherein the guide wire is coated with a PTFE.

11. A vascular access device according to claim 8, wherein the first and second annular recesses and first and second annular protrusions are generally radius ed or generally elliptical along a longitudinal direction.

12. A vascular access device comprising:
an outer sheath;
a small caliber wire inserted into a blood vessel;
means for inserting the outer sheath having a diameter of at least 5 French along the small caliber guide wire into a blood vessel.

13. A kit containing a vascular access device consisting of:
a guide wire having a diameter less or equal to 0.018 inch;
an inner cannula;
an outer sheath; and
a 21 gauge needle.

14. The kit of claim 13 wherein the inner cannula is made of metal.

15. A kit containing a vascular access device consisting of:
a guide wire having a diameter less or equal to 0.018 inch;
an inner cannula;
an outer cannula;
an outer sheath; and
a 21 gauge needle.

16. The kit of claim 15 wherein the inner cannula is made of metal.

17. A method of installing a vascular access device in blood vessel of a patient comprising the steps of:
inserting a 21 gauge needle into a blood vessel of a patient;
inserting a guide wire through the needle and partly into the blood vessel;
removing the needle;
inserting an inner cannula coaxially fitted with an outer sheath into the blood vessel until a portion of the outer sheath is within the blood vessel; and
removing the inner cannula and guide wire.

18. The method of installing a vascular access device in blood vessel of a patient according to claim 17, wherein the outer sheath is between 5 and 8 French.

19. The method of installing a vascular access device in blood vessel of a patient according to claim 17, wherein an outer cannula is coaxially disposed between the inner cannula and outer sheath.
